(19) United States
(12) Patent Application Publication
(10) Pub. No.: US 2013/0116211 A1
(43) Pub. Date: May 9, 2013

(54) Novel Salts Of Boswellic Acids And Selectively Enriched Boswellic Acids And Processes For The Same

(71) Applicants: Ganga Raju Gokaraju, Vijayawada (IN); Rama Raju Gokaraju, Vijayawada (IN); Venkata Subbaraju Gottumukkala, Tirupati (IN); Trimurtulu Golakoti, Vijayawada (IN); Venkateswarlu Somepalli, Vijayawada (IN)

(72) Inventors: Ganga Raju Gokaraju, Vijayawada (IN); Rama Raju Gokaraju, Vijayawada (IN); Venkata Subbaraju Gottumukkala, Tirupati (IN); Trimurtulu Golakoti, Vijayawada (IN); Venkateswarlu Somepalli, Vijayawada (IN)

(21) Appl. No.: 13/724,663

(22) Filed: Dec. 21, 2012

Related U.S. Application Data
(62) Division of application No. 11/817,946, filed on Aug. 14, 2008.

(51) Int. Cl. A61K 31/70; A61K 31/19; C07J 63/00; A61K 31/56

(52) U.S. Cl. 514/62; 536/55.2; 560/256; 562/498; 514/557

(57) ABSTRACT

New salts or ion-pair complexes obtained by a reaction between boswellic acids or selectively enriched 3-O-acetyl-11-keto-β-boswellic acid (AKBA) or 11-keto-β-boswellic acid (KBA) compounds obtained through a new improved process, and an organic amine, more particularly with glucosamine. These salts or ion pair complexes are useful in nutraceuticals and in food supplements for anti-inflammatory and analgesic treatment of joints and cancer prevention or cancer therapeutic agents. These salts or ion pair complexes could also be used in cosmetic or pharmaceutical composition for external treatment of body parts or organs to treat inflammatory diseases or cancer.

This invention relates to novel salts or ion pair complexes of substituted/unsubstituted boswellic acid with certain organic bases particularly though not exclusively with glucosamine. This invention also includes an improved process for selectively enriching 3-O-acetyl-11-keto-β-boswellic acid and 11-keto-β-boswellic acid hereinafter referred as (AKBA) and (KBA) respectively from an extract containing a mixture of boswellic acids.

### BACKGROUND ART

Inflammation is a critical protective biological process triggered by irritation, injury or infection, characterized by redness and heat, swelling, loss of function and pain. In addition to the foregoing induced conditions, inflammation can also occur due to age related factors. Life expectancy of general population has increased dramatically during the past few decades due to efficient control of infectious diseases and better access to nutritious food. This positive enhancement in life span coupled with changing environmental conditions elevated the incidence of chronic age-related diseases such as arthritis, diabetes, cancer, cardiovascular diseases, etc. Chronic inflammatory condition and cancer have become emerging health concerns in a number of countries across the globe and for people among all cultures. Arthritis is one of the most debilitating diseases of modem times. The quality of life for sufferers of these two diseases and their families is severely affected. Non-steroidal anti-inflammatory drugs are most commonly used remedies for rheumatic diseases. Presently, there has been a tremendous surge in demand for natural non-steroidal anti-inflammatory drugs (NSAIDs) because of their established safety and efficacy, through decades of usage by various cultures.

The inflammatory and carcinogenesis processes are known to be triggered by increased metabolic activity of arachidonic acid. Arachidonic acid diverges down into two main pathways during this process, the cyclooxygenase (COX) and lipoxygenase (LOX) pathways. The COX pathways lead to prostaglandin and thromboxane production and the LOX pathways leads to leukotrienes (LTS) and hydroxyl eicosatetetraenoic acid (HETEs). These classes of inflammatory molecules exert profound biological effects, which enhance the development and progression of human cancers,

Leukotrienes and 5(S)-HETE are important mediators for inflammatory, allergic and obstructive process. Leukotrienes increase micro vascular permeability and are potent chemotactic agents. Inhibition of 5-lipoxygenase indirectly reduces the expression of TNF-α (a cytokine that plays a key role in inflammation). 5-Lipoxygenase is therefore the target enzyme for identifying inhibitors, which have potential to cope with a variety of inflammations and hypersensitivity-based human diseases including asthma, arthritis, bowel diseases such as ulcerative colitis and circulatory disorders such as shock and ischaemia.

Similarly prostaglandins are intercellular messengers that are produced in high concentration at the sites of chronic inflammation and are capable of causing vasodilation, increased vascular permeability and sensitizing pain receptors. The pro-inflammatory prostaglandins (PGE2) are produced by inducible isoform cyclooxygenase-2 (COX-2). The prostaglandins that are important in gastrointestinal and renal function are produced by the constitutively expressed isoform, cyclooxygenase-1 (COX-1). COX-1 is the protective housekeeper isoform and it regulates mucosal cell production of mucous that provides a barrier between the acid and pepsin present in gastric secretions. Non-selective COX inhibitors thus produce serious side effects. Scientists around the world are thus investing a major effort in identifying non-steroidal anti-inflammatory drugs that inhibit 5-lipoxygenase and cyclooxygenase-2 enzymes. As both COX-2 and 5-LOX are commonly expressed in any kind of inflammatory condition, efforts are currently being focused to obtain the so called dual acting anti-inflammatory drugs that are able to inhibit both COX-2 and 5-LOX enzymes. Unfortunately, the odds of finding a new dual acting natural NSAID that can truly alleviate the symptoms of inflammatory diseases are very thin. Hence, the researchers conceived the idea that using a combination of drugs, one having the COX-2 inhibitory activity and the other having 5-LOX inhibitory activity, as the next best option.

Rheumatoid arthritis is a chronic inflammatory condition that affects the lubricating mechanism and cushioning of joints. As a result of this autoimmune disease the bone surfaces are destroyed, which leads to stiffness, swelling, fatigue and crippling pain. Osteoarthritis is the common form of arthritis and results primarily from progressive degeneration of cartilage glycoaminoglycons. The damage is often compounded by a diminished ability to restore and repair joint structures including cartilage. The smooth surface of the cartilage becomes hard and rough creating friction. As a result of this the joint gets deformed, painful and stiff. Studies have indicated that over 40 million Americans have osteoarthritis, including 80% of persons over the age of 50. The major focus for osteoarthritis treatment, should therefore involve agents that not only stimulate the production of biological substances necessary for regeneration of cartilage cells and proper joint function but also diminish pain inflammation.

It is therefore an object of the present invention to provide a salt or ion pair complex as a dietary supplement, that exhibits anti-arthritic properties without deleterious side effects.

Boswellic Acids

Gum resin of Boswellia species known as Indian frankincense has been used as an anti-inflammatory agent in Traditional Ayurvedic Medicine in India. Ancient Ayurvedic texts described its therapeutic use. Clinical trails performed by CSIR laboratories in India have shown fair to excellent results in 88% of the patients, with no adverse side effects (Singh, G. B., Status report, anti-inflammatory drugs from plant sources, 1982). A randomized, double blind, placebo controlled clinical trials on patients with osteo-arthritis of knee exhibited statistically significant improvement in the pain, decreased swelling and increased knee flexion etc. (Kimmatkar, Phytomedicine, 2003, 10, 3-7), The therapeutic effects shown by Boswellia serrata extract were comparable to those exhibited by sulfasalazine and mesalazine in patients with ulcerative colitis. (Gupta, I., et al., Eur. J. Med. Res., 1998, 3, 511-14 and Gerhardt, H., et. al., Gastroenterol., 2001, 39, 11-17). The source of anti-inflammatory actions has been attributed to boswellic acids (Safayhi, H., et al., Planta Medica, 1997, 63, 487-493 and J. Pharmacol. Exp. Ther., 1992, 261, 1143-46, both the journals published from USA), a group of triterpene acids isolated from the Boswellia resin (Pardhy, R. S., et al., Indian J. Chem., 1978, 16B, 176-178). These compounds exert anti-inflammatory activity by inhibiting 5-lipoxygenase (5-LOX). The boswellic acids also gained prominence recently for their antiproliferative actions. Boswellic acids inhibited several leukemia cell lines in vitro and inhibited melanoma growth and induced apoptosis (Hostanska, K., et al., Anticancer Res., 2002, 22(5), 2853 -62). The acetyl boswellic acids were found to be unique class of dual inhibitors of human topoisomerases I and II a (Syrovets, T. et al., Mol. Pharmacol., 2000, 58(1), 71-81). Immunomodulatory activity of boswellic acids had been reported by Sharma et al. in Phytotheraphy Research, 1996, 10, 107-112, published from USA. A detailed study on the structural requirements for boswellic acids indicated that of all the six acids, 3-O-acetyl-11-keto-β-boswellic acid, hereinafter referenced as AKBA shows most pronounced inhibitory activity against 5-LOX (Sailer, E. R., et al., British J. Pharmacology, 1996, 117, 615-618). AKBA acts by unique mechanism, in which it binds to 5-LOX in a calcium-dependent and reversible manner and acts as a non-redox-type, non-competitive inhibitor (Sailer, E. R., et al., Eur. J. Biochem., 1998, 256, 364-368). The AKBA or a plant extract or composition containing it was reported to be effective for topical application, as an agent to soften lines and/or relax the skin (Alain, M., et. al., U.S. patent application No. 2004/0166178, dated Aug. 26, 2004). AKBA has thus become the subject of intensive research for its potential for the treatment of chronic inflammatory disorders.

Glucosamine

Glucosamine is a natural substance found in high quantities in joint structures. The main function of glucosamine in joint structures is to produce cartilage components necessary for maintaining and repair joint tissue. Glucosamine stimulates the formation of joint structural components such as collagen, the protein of the fibrous substances that holds the joints together and helps to build-up the cartilage matrix, Collagen is the main component of the shock-absorbing cushion called articular cartilage. It is also a necessary nutrient in the production of synovial fluid. Some people may lose the ability with age to produce glucosamine, thereby inhibiting the grpwth of cartilage destroyed during wear and tear in osteoarthritis patients (Towheed, T. E., Arthritis and Rheumatism, 2003, 49, 601-604). When taken orally as a dietary supplement in the form of glucosamine sulfate, it has been shown to exert protective effect against joint destruction and is selectively used by joint tissues to promote healthy joint function and show potential therapeutic effect in osteoarthritis (Perry, G. H., et al., Ann. Rheum. Dis., 1972, 31, 440-448).

Several double-blind studies with glucosamine sulfate showed therapeutic effects comparable to or even better than non steroidal anti-inflammatory drugs in relieving the symptoms of osteoarthritis (Vaz, A. L., Curr. Med, Res. Opin., 1982, 8, 145-149; D'Ambrosia, E. D., et al., Pharmatherapeutica, 1982, 2, 504-508 and Tapadinhas, M. J., et al., Pharmatherapeutica, 1982, 3, 157-168). The NSAIDs offer only symptomatic relief, whereas glucosamine addresses the root cause of osteoarthritis disease. It support body's natural ability to tackle the disease on its own by providing the building blocks to many structural components such as glucosaminoglycons to repair the damage caused by osteoarthritis. Glucosamine hydrochloride is used for this study.

### DISCLOSURE OF THE INVENTION

The organic solvent extract of the gum resin of Boswellia serrata contain a total of six boswellic acids and two timcallic acids. These acids are shown in FIG. 1, and are represented by B1, B2, B3, B4, B5, B6, T1, T2 and T3. Studies have indicated AKBA as the most potent an anti-inflammatory agent among all the boswellic acids. The concentration of AKBA, indicated as B2 in the FIG. 1, amounts only in the range of 1-10% in the extract, but most often it is in the range of 2-3%. The potential usefulness of boswellic acids in general and AKBA in particular can be a great incentive to take-up further development of these compounds in all possible aspects.

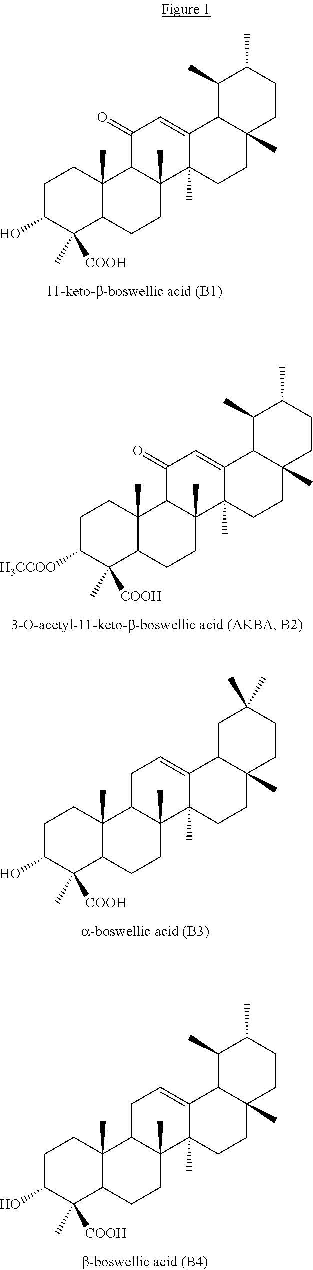

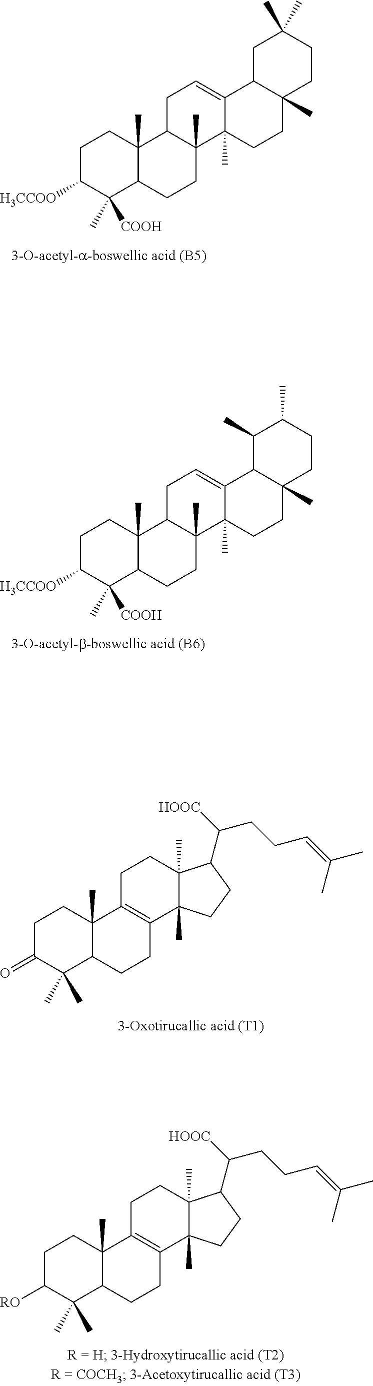

The present invention is aimed at selective enrichment of active compounds, KBA and AKBA to a therapeutically useful range such as 30% to 100% from natural Boswellia extract using a new improved process and then converting the enriched compounds to a salt or ion pair complex with enhanced solubility and improved therapeutic efficacy for use as an anti-arthritic dietary supplement. The salt or ion pair combination may be accomplished by using an acid function of the boswellic acid and an amine function from amino organic compounds, especially glucosamine.

The enrichment of AKBA from natural Boswellia extract was already described in international patent application (PCT #WO 03/074063, dated. 12th September 2003) and also in US patents (application #20030199581, publication dated 23 Oct. 2003 and application #20040073060, publication dated 15 Apr. 2004). The processes described in these patents involve multi-step procedures and requires tedious work-up and chromatographic purifications. The present invention is an improved method, where in the acetylation and allylic oxidation steps are conducted in a single pot. This process eliminates the need for labor-intensive work-up following acetylation and time consuming product drying before proceeding to the oxidation step, as required by the processes reported in the patents and journal articles. This process also efficiently utilizes the un-reacted pyridine and acetic anhydride from the acetylation step to form highly active oxidizing systems such as CrO₃/pyridine and CrO₃/acetic anhydride. The present invention effectively reduces the overall reaction time for peracetylation and the oxidation steps. The new process eliminates the presence of possible chromium impurities in the KBA/AKBA enriched (30-40%) product by acid/base treatment without any need for chromatography.

A fraction enriched to 30-40% 11-keto-β-boswellic acid (KBA), can be accomplished by subjecting the crude mixture to basic treatment, followed by filtration and acidification of the mother liquor, and then separation of the white solid by filtration and drying, It was then reacetylated to obtain 30-40% AKBA enriched fraction, The fractions enriched to higher percentage (40-100%) of KBA and AKBA can be obtained by applying chromatographic methodology on hydrolysis mixture and re-acetylation mixture, respectively.

An ionic salt or ion pair complex of boswellic acids containing AKBA in the range of 5 to 100% can be obtained by using appropriately enhanced boswellic compound and a suitable amine compound and adopting the representative procedure given in the examples.

This invention relates to novel salts or ion pair complexes of boswellic acid and keto boswellic acid and acetyl keto boswellic acid with glucosamine having the following general formula.

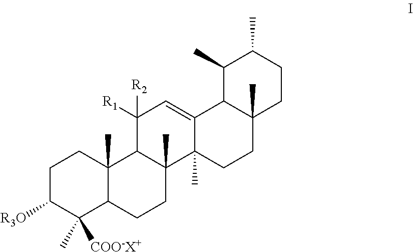

wherein R₁ and R₂ are H or taken together to form a keto group;
R₃ is H or acyl group;
X is an heterocyclic base or an organic bases represented by NHR₄R₅R₆:
wherein R₄, R₅ and R₆, are H or substituted or unsubtituted lower or higher
alkyl group or aryl group or cyclic alkyl group.

Wherein the organic bases are glucosamine (2-amino-2-deoxy-D-glucose), nicotinamide (3-pyridinecarboxamide), pyridoxins (5-hydroxy-6-methyl-3,4-pyridinedimethanol), caffeine (3,7-dihydro-1,3,7-trimethyl-1H-purine-2,6-dxone), creatine (N-(aminoiminomethyl)-N-methylglycine), allantoin (2,5-dioxo-4-imidazolidinyl)urea), Theobromine (3,7-dihydro-3,7-dimethyl-1H-purine-2,6-dione), theophylline (3,7-dihydro-1,3-dimethyl-1H-purine-2,6-dione), mesalamine (5-amino-2-hydroxybenzoic acid), enfenamic acid (2-[(2-phenylethyl)amino]benzoic acid), etofenamate (2-[[3-(trifluoromethyl)phenyl]-amino]benzoic acid 2-(2-hydroxyethoxyethyl ester), flufenamic acid (2-[[3-(trifluoromethyl)phenyl]amino]benzoic acid), meclofenamic acid (2-[(2,6-dichloro-3-methylphenyl)amino]benzoic acid), mefenamic acid (2-[(2,3-dimethylphenyl)-amino]benzoic acid), niflumic acid (2-[[3-(trifluoromethyl)phenyl]-amino]-3-pyridinecarboxylic acid), talniflumate (2-[[3-(trifluoromethyl)phenyl]amino]-3-pyridinecarboxylic acid 1,3-dihydro-3-oxo-1-isobenzofuranyl ester), terofenamate (2-[(2,6-dichloro-3-methylphenyl)-amino]benzoic acid ethoxymethyl ester), tolfenamic acid (2-[(3-chloro-2-methylphenyl)-amino]benzoic acid), S-adenosylmethionine ((3S)-5′-[(3-amino-3-carboxypropyl)methylsulfonio]-5′-deoxyadenosine inner salt), 3-amino-4-hydroxybutyric acid, amixetrine (1[2-(3-methylbutoxy)-2-phenylethyl]pyrrolidine), benzydamine (N,N-dimethyl-3-[[1-(phenylmethyl)-1H-indazol-3-yl]oxy]-1-propanamine), difenpiramide (N-2-pyridinyl-[1,1′-biphenyl]-4-acetamide), ditazol (2,2′-[(4,5-diphenyl-2-oxazolyl)imino]-bisethanol), emorfazone (4-ethoxy-2-methyl-5-(4-morpholinyl)-3(2H)-pyridazinone), fepradinol ((±)-α-[[(2-hydroxy-1, 1-dimethylethyl)-amino]methyl]benzeneniethanol), paranyline (4-(9H-fluoren-9-ylidenemethyl)benzene-carboximidamide), perisoxal (α-(5-phenyl-3-isoxazolyl)-1-piperidineethanol).

We have disclosed a simple method by which salts or ion-pair complexes of boswellic acids with hetero-atom bases (also referred to as ‘organic base’) can be made for their inclusion in dietary or pharmaceutical compositions that provide reduction in inflammation and other health benefits. These salts or ion pair complexes are made by simple acid-base reaction, as shown in eq, 1, between an organic acid (RCOOH) and an organic base (NR₄R₅R₆).

RCOOH+NR₄R₅R₆→RCOO⁻⁺NHR₄R₅R₆ (equation 1)

The new composition according to this invention may be prepared by the following processes:
(a) By reacting boswellic acids or ketoboswellic acid or acetyl ketoboswellic acid with organic base.
(b) By in situ generation of organic free base and reacting with boswellic acids or ketoboswellic acid or acetyl ketoboswellic acid.

In the first process, stoichiometric equivalents of the reactants are mixed to obtain the desired salts or ion pair complexes. Preferably, the reaction is initiated by the slow addition of organic free base, particularly, glucosamine free base to an aqueous methanolic solution of boswellic acids. Boswellic acids (48% by HPLC) may be obtained by a known process of extraction from the gum resin of Boswellia serrata. Glucosamine free base may be liberated from glucosamine hydrochloride by anionic exchange resin treatment. The enriched 11-ketoboswellic acid or 3-O-acetyl-11-ketoboswellic acid (30%-100%) was obtained from the gum-resin of Boswellia serrata using an improved method described herein.

The salts or pair complexes prepared by this process may contain between 10 to 70% of boswellic acids. 5-40% of glucosamine.

According to the second process of preparing the compounds of this invention, stoichiometric quantities of boswellic acids, potassium hydroxide and organic base salts, particularly, glucosamine hydrochloride are reacted in aqueous methanol medium.

A further aspect of the present invention is a pharmaceutical formulation comprising a compound as described above in a pharmaceutically acceptable carrier (e.g., an aqueous or a non aqueous carrier).

A still further aspect of the present invention is a method of treating inflammatory diseases, comprising administering to a human or animal subject in need thereof a treatment effective amount (e.g., an amount effective to treat, slow the progression of, etc.) of a compound as described above.

Preferred embodiments relating to the improved process of enriching AKBA in natural Boswellia extract and making the salts or ion pair complexes are presented in examples 1 to 6.

Though the following examples describe a specific embodiment of this invention, obvious equivalents and modifications known to persons skilled in the art are not excluded from the scope of the appended claims.

### EXAMPLE 1

### Isolation of 11-keto-β-boswellic acid and 3-O-acetyl-11-keto-β-boswellic acid

1a). Single Pot Conversion of Boswellia Extract into AKBA Enriched Fraction:

To a solution of Boswellia serrata extract (85%, 10 kg,) in pyridine (5.4 L), in a 100 L all glass reactor equipped with a water-cooled reflux condenser, was added acetic anhydride (4.2 L) at room temperature and the mixture was subjected to heating at 60-65° C. under stirring. After 3 h, the mixture was cooled to ambient temperature and diluted with acetic acid (24 L) and acetic anhydride (24 L). The contents were cooled and treated slowly with chromium trioxide (6.4 kg) while maintaining the temperature under 40° C. The stirring was continued for another 2 h after the addition, and then the mixture was poured into ice water and the contents were mixed thoroughly. The solid was filtered, washed with water and dried in a vacuum oven to obtain a residue (14 kg). The HPLC analysis of the crude product showed complete conversion of boswellic acids B1, B4 and B6 to B2 (AKBA).

1b). Isolation of 30-40% 3-O-acetyl-11-keto-β-boswellic acid: The above crude reaction mixture (5 kg) was added to 4N hydrochloric acid (45 L) and heated at 60° C. for 4 h. The mixture was cooled to ambient temperature and filtered. The precipitate was washed with 4N HCl, followed by water and dried in a vacuum oven to obtain AKBA enriched to 30-40% (2.8 kg).

1c). Isolation of 3-O-acetyl-11-keto-β-boswellic acid: The above, enriched compound (500 g) was subjected to silica column chromatography using 5% to 30% ethyl acetate/hexane mixtures. The fractions were monitored by TLC and those containing AKBA (30%-60%) were combined and subjected crystallization in hexane and ethyl acetate mixtures to obtain fractions enriched up to 85% AKBA, Repeated crystallization in the same solvent system yielded AKBA enriched up to 100%.

1d). Isolation of 11-keto-β-bowellic acid: Alternatively, the crude mixture was dissolved in methanol and subjected to base treatment (8N KOH), The precipitate was separated by filtration and discarded. The mother liquor was acidified and the off-white precipitate was filtered, washed with water and dried under vacuum to obtain 30-40% ketoboswellic acid (KBA). The 11-keto-β-boswellic acid mixture (200 g) obtained was adsorbed on 250 g of silica gel and subjected column chromatography over 500 g of silica. The column was eluted with hexane, 10% ethyl acetate/hexane, 20% ethyl acetate/hexane and 30% ethyl acetate/hexane mixtures. The fractions were monitored by TLC and the fractions containing 11-keto-p-boswellic acid were combined and evaporated and the residue was subjected to repeated crystallization from ethyl/hexane mixtures to obtain pure 11-keto-β-boswellic acid (45 g, 95-100%. purity).

1e). In a further variation of the process mentioned in example 1a, the addition of acetic anhydride was eliminated. Instead the peracetylated mixture was diluted with 20 L of acetic acid and treated with CrO₃ (6.4 kg) in 100 L of acetic acid. The reaction mixture was quenched with excess water after 24 h, and processed as described in example 1a,

### EXAMPLE 2

Glucosamine salt of boswellic acids; To a solution of boswellic acids (2 g, 48% boswellic acids) in 95% aqueous methanol (50 mL) was added glucosamine free base solution (8.6 mL, 0.4 g) and stirred at rt for 1 h. Then the solvent was evaporated under reduced pressure and dried to give glucosamine salt or ion pair complex of boswellic acids as gray colour powder (2.3 g), pH, 6,3, soluble in 90% aqueous methanol.

The analytical characteristics of the glucosamine salt or ion pair complex of boswellic acids thus obtained are, B1, 4.75%, B2, 2,10%, B3, 5.44%, B4, 14.91%, B5, 2.18%, B6, 8.66%; total: 38.04%; glucosamine (as free base) is 8.52%.

### EXAMPLE 3

Glucosamine salt of boswellic acids (KCl): To a solution of boswellic acids (5 g, 48% boswellic acids) in methanol (125 mL) was added a solution of glucosamine hydrochloride (2 g) in water (8 mL) and stirred at rt for 15 min. Then potassium hydroxide (0.52 g, 20% aqueous solution, 2.6 mL) was charged slowly for 10 min and the solution was stirred at rt for 1 h. The solvent was evaporated under reduced pressure and dried to give glucosamine salt or ion pair complex of boswellic acids as gray colour powder (7.5 g), pH, 6.8, soluble in 90% aqueous methanol.

The analytical characteristics of the glucosamine salt or ion pair complex of boswellic acids thus obtained are, B1, 4.04%, B2, 1.86%, B3, 4.65%, B4, 12.73%, B5, 1.76%, B6, 7.34%; total: 32.38%; glucosamine (as free base) is 12.44%.

### EXAMPLE 4

Glucosamine salt of boswellic acids (KCl): To a solution of boswellic acids (5 g, 48% boswellic acids) in methanol (125 mL) was added a solution of glucosamine hydrochloride (4g) in water (11 mL) and stirred at rt for 15 min. Then potassium hydroxide (0.52 g, 20% aqueous solution, 2.6 mL) was charged slowly for 10 min and the solution was stirred at rt for 1 h. The solvent was evaporated under reduced pressure and dried to give glucosamine salt or ion pair complex of boswellic acids as gray colour powder (9.6 g), pH, 6.6, soluble in 90% aqueous methanol.

The analytical characteristics of the glucosamine salt or ion pair complex of boswellic acids thus obtained are, B1, 3.14%, B2, 1.37%, B3, 3.36%, B4, 9.75%, B5, 0.93%, B6, 4.76%; total: 23.31%; glucosamine (as free base) is 27.16%.

### EXAMPLE 5

Glucosamine salt of Acetyl ketoboswellic acid (KCl); To a solution of acetyl ketoboswellic acid (5 g, 30% AKBA) in methanol (100 mL) was added a solution of glucosamine hydrochloride (0.63 g) in water (3 mL) and stirred at rt for 15 min. Then potassium hydroxide (0.164 g, 20% aqueous solution, 0,82 mL) was charged slowly for 10 min and the solution was stirred at rt for 1 h. The solvent was evaporated under reduced pressure and dried to give glucosamine salt or ion pair complex of acetyl ketoboswellic acid as gray colour powder (4.8 g), pH, 6.7, soluble in 90% aqueous methanol.

The analytical characteristics of the glucosamine salt or ion pair complex of acetyl ketoboswellic acid thus obtained are, AKBA is 27.68%; glucosamine (as free base) is 5.42%.

### EXAMPLE 6

Glucosamine salt of Acetyl ketoboswellic acid (KCl): To a solution of acetyl ketoboswellic acid (5 g, 30% AKBA) in methanol (100 mL) was added a solution of glucosamine hydrochloride (5 g) in water (15 mL) and stirred at rt for 15 min. Then potassium hydroxide (0.2 g, 20% aqueous solution, 1.0 mL) was charged slowly for 10 min and the solution was stirred at rt for 1 h. The solvent was evaporated under reduced pressure and dried to give glucosamine salt or ion complex of acetyl ketoboswellic acid as gray colour powder (9.3 g), pH, 5.6, soluble in 90% aqueous methanol.

The analytical characteristics of the glucosamine salt or ion pair complex of acetyl ketoboswellic acid thus obtained are, AKBA is 15.30%; glucosamine (as free base) is 39.44%.

1.-6. (canceled)

7. A process for the preparation of a salt or ion-pair complex containing boswellic acids and glucosamine having the general formula I 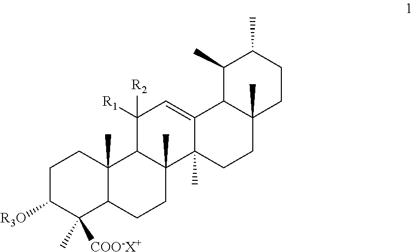
wherein R₁ and R₂ are H or taken together to form a keto group;
R₃ is H or acyl group;
X is a residue of glucosamine;
comprising the step of slowly adding glucosamine free base to an aqueous methanolic solution of boswellic acids.

8. The process as claimed in claim 7 wherein said salt or ion-pair complex is recovered from the reaction mixture by removing solvent under reduced pressure.

9. A process for the preparation of a salt or ion-pair complex containing boswellic acids and glucosamine of the general formula I 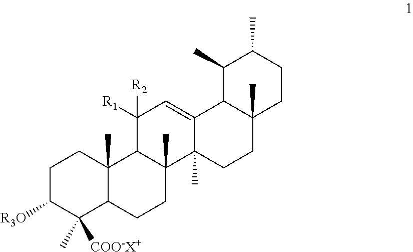
wherein R₁ and R₂ are H or taken together to form a keto group;
R₃ is H or acyl group;
X is residue of glucosamine;
comprising the steps of in situ generation of glucosamine free base and reacting with boswellic acids.

10. The process as claimed in claim 9, wherein said in situ generation of free base is accomplished by the addition of bases like potassium hydroxide, sodium hydroxide and is carried out in the presence of alcohols or hydroalcohols.

11. The process as claimed in claim 9, wherein said salt or ion-pair complex is obtained by evaporating the solvent under reduced pressure.

12.-14. (canceled)

15. A process for producing 25-100% 3-O acetyl-11-keto-β-boswellic acid, from an extract containing a mixture of boswellic acids obtained from gum resin of Boswellia species comprising the steps of:
acetylating a boswellic acids containing fraction from said extract in a reaction vessel to form an aceiylated product;
oxidizing the acetylated product in the reaction vessel, without any intermediate work-up of the acetylated product, to form an aceiylated and oxidized product;
acid treating and/or using chromatographic separation on the acetylated and oxidized product to obtain a fraction enriched in 3-O acetyl-11-keto-β-bosvvellic acid in a range of about 25-100%.

16. (canceled)

17. A process for producing 25-100%11-keto-β-boswellic acid from gum resin of Boswellia species comprising the steps of:
acetylating a boswellic acids containing fraction from said extract in a reaction vessel to form an acetylated product;
oxidizing the acetylated product in the reaction vessel, without any intermediate work-up of the acetylated product, to form an acetylated and oxidized product;
base treating and/or using chromatographic separation on the acetylated and oxidized product to obtain a fraction enriched in 11-keto-β-boswellic acid in a range of about 25-100%.

18. The process claimed in claim 15, wherein, the acetylation step is carried out by treating the said extract with acetic anhydride/pyridine or acetyl chloride/pyridine or acetic anhydride alone.

19. The process claimed in claim 15, wherein the oxidation is carried out by treating the acetylated product with acetic acid, acetic anhydride/chromium trioxide or acetic acid/chromium trioxide.

20. The process claimed in claim 15, the acetylated and oxidized product is formed by pouring into ice-water followed by filtration of the solid, washing with water and drying.

21. The process claimed in claim 15, wherein the acetylated and oxidized product is treated with 4N HCl at elevated temperatures and the resulting mixture is filtered, washed with 4N HCl and water and dried to obtained 25-40% AKBA.

22. The process claimed in claim 21, wherein the said product after add treatment is subjected to silica column chromatography using organic solvents as selected from the group consisting of acetone, chloroform, dichloromethane, ethyl acetate, hexane, methanol and mixtures-thereof to obtain an AKBA enriched fraction, which after repeated crystallization in a suitable solvents yields 40-100% AKBA.

23. The process claimed in claim 17, wherein the acelylated and oxidized product is subjected to base (8N KOH) treatment in a suitable solvent, followed by filtration and acidification of a mother liquor, filtration of a solid and drvine to obtain 25-40% KBA.

24. The process claimed in claim 23, wherein the said product after base treatment is subjected to silica column chromatography using organic solvents as selected from the group consisting of acetone, chloroform, dichloromethane, ethyl acetate, hexane, methanol and mixtures thereof to obtain a KBA enriched fraction, which after repeated crystallization in a suitable solvent yields 40-100% KBA.

25. The process as claimed in claim 22, wherein the solvent of crystallization is acetone, chloroform, dichloromethane, ethyl acetate, hexane and methanol either alone or in combination.

26. The process as claimed in claim 15, wherein the KBA enriched in the range of 25-100% is acetylated using pyridine/acetic anhydride or acetic anhydride alone to obtain AKBA enriched in the range of 25-100%.

27.-29. (canceled)

30. A method of treating inflammatory diseases comprising administrating a salt or ion-pair complex of the formula I to a person in need thereof 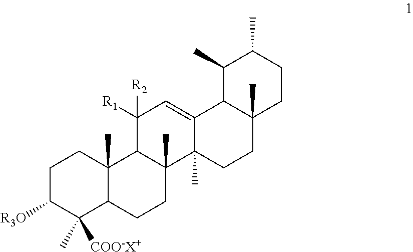
wherein R₁ and R₂ are H or taken together to form a keto group;
R₃ is H or acvl group;
X is a residue of a base selected from the group consisting of a heterocyclic base and an organic base represented by NHR₄R₅R₆ and mixtures thereof;
wherein R₄, R₅ and R₆, are H substituted or unsubstituted lower or higher alkyl group or aryl group or cyclic alkyl group,
said organic base is glucosamine (2-amino-2-deoxy-D-glucose, nicotinamide (3-pyridinecarboxamide), pyridoxine (5-hydroxy-6-methyl-3,4-pyridinedimethanol), caffeine (3,7-dihydro-1,3,7-trimethyl-1H-purine-2,6-dione), creatine (N-(aminoiminomethyl)-N-methylglycine), allantoin (2,5-dioxo-4-imidazolidinyl)urea), Theobromine (3,7-d.ihydro-3,7-dimethyl-1H-purine-2,6-dione), theophylline (3,7-dihydro-1,3-dimethyl-1H-purine2,6-dione), mesalamine (5-amino-2-hydroxybenzoic acid), enfenamic acid (2-[(2-phenylethyl)amino]benzoic acid), etofenamate (2-[[3-(trifluoromethyl)phenyl]-amino]benzoic acid 2-(2-hydroxyethoxyethyl ester), flufenamic acid (2-[[(3(trifluoromethyl)phenyl]amino]benzoic acid), meclofenamic acid (2-[(2,6-dichloro-3-methylphenyl)amino]benzoic acid), mefenamic acid (2-[(2,3-dimethyiphenyl)-amino]bertzoic acid), niflumic acid (2-[[3-(trifluoromethyl) phenyl]-amino]-3-pyridinecarboxylic acid), talniflumate (2-[[3-(trifluoromethyl)phenyl]amino]-3-pyridinecarboxylic acid 1,3-dihydro-3-oxo-1-isobenzofuranyl ester), terofenamate (2-[(2,6-dichloro-3-methylphenyl)-amino]benzoic acid ethoxymethyl ester), tolfenamic acid (2-[(3-chloro-2-methylphenyl)-amino]benzoicacid), S-adenosylrnethionine ((3S)-5′-[(3-amino-3-carboxypropyl)methylsulfonio]-5′-deoxyadenosine inner salt), 3-amino-4-hydroxy butyric acid, amixetrine (1-[2-(3-methylbutoxy)-2-phenylethyl]pyrrolidine), benzydamine (N,N-dimefhyl-3-[[1-(phenylmethyl)-1H-indazol-3-yl]oxy]-1-propanamine), difenpiramide (N-2-pyridinyl-[1.1′-biphenyl]-4-acetamide), ditazol (2,2′-[(4,5-diphenyl-2-oxazolyl)imino]-bisethanol), emorfazone (4-ethoxy-2-methyl-5-(4-morpholinyl)-3(2H)-pyridazinone), fepradinol ((±)-α-[[(2-hydrixt-1,1-dimethylethyl)-amino]methyl]benzenemethanol), paranyline (4-(9H-fluoren-9-ylidenemethyl)benzene carboximidamide), or perisoxal (α-5-phenyl-3-isoxazolyl)-1-piperidineethanol).

31.-32. (canceled)

33. A method of producing a salt or ion-pair complex of an acid and an organic amine comprising the steps of:
acetylating a boswellic acids containing fraction from a Boswellia extract in a reaction vessel to form an acetylated product;
oxidizing the acetylated product in the reaction vessel, without any intermediate work-up of the acetylated product, to form an acetylated and oxidized product;
treating the acetylated and oxidized product with at least one of an acid treatment, a base treatment and chromatographic separation to form an enriched product containing at least one of an enriched keto boswellic acid (KBA), enriched acetyl ketoboswellic acid (AKBA) and mixtures thereof, the enriched KBA being a part of a blend having a concentration of KBA which is above the concentration of KBA in a gum resin obtained from. Boswellia serrata and the enriched AKBA being part of a blend having a concentration of AKBA which is above the concentration of AKBA in a gum resin obtained from Boswellia serrata; and
combining an organic base with the enriched product in a methanol solution to form the salt or ion-pair complex having the following general formula I, 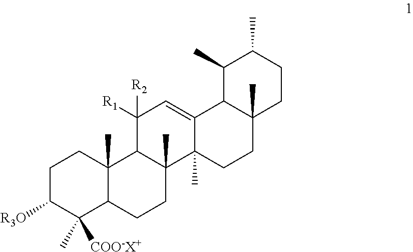
wherein R₁ and R₂ are H or taken together to form a keto group;
R₃ is H or acyl group;
X is a residue of a base selected from the group consisting of a heterocyclic base and an organic base represented by NHR₄R₅R₆ and mixtures thereof;
wherein R₄ R₅ and R₆, are H substituted or unsubsrituted lower or higher alkyl group or aryl group or cyclic alkyl group,
said organic base is glucosamine (2amino-2-deoxy-D-glucose), nicotinamide (3-pyridinecarboxamide), pyridoxine (5-hydroxy-6-methyl-3,4-pyridinedimethanol), caffeine (3,7-dihydro-1,3,7-trimethyl-1H-purine-2,6-dione), creatine (N-(aminoiminomethyl)-N-methylglycine), allantoin (2,5-dioxo-4-imidazolidinyl)urea), Theobromine (3,7-dihydro-3,7-dimethyl-1H-purine-2,6-dione), theophylline (3,7-dihydro-1,3-dimethyl-1H-purine-2,6-dione), mesalamine (5-amino-2-hydroxybenzoic acid), enfenamic acid (2-[(2-phenylethyl)amino]benzoic acid), etofenamate (2-[[3-(trifluoromethyl)phenyl]-amino]benzoic acid 2-(2-hydroxyethoxyethyl ester), flufenamic acid (2-[[3-(trifluoromethyl)phenyl]amino]benzoic acid), meclofenamic acid (2-[(2,6-dichloro-3-methylphenyl)amino]benzoic acid), mefenamic acid (2-[(2,3-dimethylphenyl)-amino]benzoic acid), niflumic acid (2-[[3-(trifluoromethyl) phenyl]-amino]-3-pyridinecarboxylic acid), talniflumate (2-[[3-(trifluoromethyl)phenyl]amino]-3-pyridinecarboxylic acid 1,3-dihydro-3-oxo-1-isobenzofixraiwl ester), terofenamate (2-[(2,6-dichloro-3-methylphenyl)-amino]benzoic acid ethoxymethyl ester), tolfenamic acid (2-[(3-chloro-2-methylphenyl)-amino]benzoic acid), S-adenosylmethionine ((3S)-5′-[(3-amino-3-carboxypropyl)methylsulfonio]-5′-deoxyadenosine inner salt), 3-amino-4- hydroxy butyric acid, amixetrine (1-[2-(3-methylbutoxy)-2-phenylethyl]pyrrolidine), benzydamine (N,N-dimethyl-3-[[1-(phenylmethyl)-1H-indazol-3-yl]oxy]-1-propanamine), difenpiramide (N-2-pyridinyl-[1,1′-biphenyl]-4-acetamide), ditazol (2,2′-[(4,5-dipheyl-2-oxazolyl)imino]-bisethanol), emorfazone (4-ethoxy-2-methyl-5-(4-morpholinyl)-3(2H)-pyridazinone), fepradinol ((±)-α-[[(2-hydroxy-1,1-dimethylethyl)-amino]methyl]benzenemethanol), paranyline (4-(9H-fluoren-9-ylidenemethyl)benzene carboximidamide), or perisoxal (α-(5-phenyl-3-isoxazolyl)-1-piperidinethanol).
